(19) United States
(12) Patent Application Publication
(10) Pub. No.: US 2026/0014930 A1
(43) Pub. Date: Jan. 15, 2026

(54) WHEEL SYSTEM AND METHOD FOR INDICATING STATUS OF DRIVER

(71) Applicant: COPLUS INC., Tainan City (TW)

(72) Inventor: Po-Hua WU, Tainan City (TW)

(73) Assignee: COPLUS INC., Tainan City (TW)

(21) Appl. No.: 19/263,018

(22) Filed: Jul. 8, 2025

(30) Foreign Application Priority Data
Jul. 10, 2024 (TW) 113125828

(51) Int. Cl. B60Q 1/50; A61B 5/0205; A61B 5/024; A61B 5/05; A61B 5/08; B60B 3/10; B60Q 1/32; G01N 33/00; G06V 20/59

(52) U.S. Cl. CPC B60Q 1/544; A61B 5/0205; A61B 5/05; B60B 3/10; B60Q 1/326; B60Q 1/543; G01N 33/0036; G01N 33/0063; G06V 20/597; A61B 5/02444; A61B 5/0816; A61B 2503/22; B60B 2900/571; B60B 2900/572; B60Q 2400/20; B60Q 2900/30

(57) ABSTRACT

A wheel system for a vehicle body includes a control device and a wheel assembly. The control device stores multiple warning conditions, and multiple warning parameters corresponding respectively to the warning conditions, and is configured to detect a target object in the vehicle body so as to generate sensing data. The control device is further configured to, when one of the warning conditions is met based on the sensing data, obtain a corresponding one of the warning parameters that corresponds to the one of the warning conditions, and output a control signal that includes the corresponding one of the warning parameters. The wheel assembly is configured to receive the control signal from the control device, to emit light with one of multiple color patterns which corresponds to the corresponding one of the warning parameters included in the control signal, and to generate electric power.

### CROSS-REFERENCE TO RELATED APPLICATION

This application claims priority to Taiwanese Invention patent application Ser. No. 11/312,5828, filed on Jul. 10, 2024, the entire disclosure of which is incorporated by reference herein.

### FIELD

The disclosure relates to a wheel, and more particularly to a wheel system and a method for indicating a status of a driver of a vehicle.

### BACKGROUND

As technology advances, more functions are available in a vehicle for improving driving safety, including distance measurement systems, oncoming traffic warnings, etc. However, conventional designs for these systems often rely on sensing devices that are mounted on the vehicle to detect other vehicles or the surrounding environment to generate warnings. As a result, if the vehicle itself is abnormal, surrounding vehicles (e.g., other vehicles behind the abnormal vehicle) often have insufficient time to react, potentially leading to a traffic accident.

### SUMMARY

Therefore, an object of the disclosure is to provide a wheel system that can alleviate at least one of the drawbacks of the prior art.

According to an aspect of the disclosure, a wheel system for a vehicle body is provided, and includes a control device and a wheel assembly. The control device includes a first wireless communication module, a sensing module and a warning control module. The sensing module is configured to detect a target object in the vehicle body so as to generate sensing data. The warning control module stores a plurality of warning conditions, and a plurality of warning parameters corresponding respectively to the warning conditions. The warning control module is configured to, in response to determining that one of the warning conditions is met based on the sensing data, obtain a corresponding one of the warning parameters that corresponds to the one of the warning conditions, and output a control signal that includes the corresponding one of the warning parameters through the first wireless communication module. The wheel assembly is adapted to be mounted on the vehicle body and includes a wheel, a lighting module, a light control module and a power generating mechanism. The lighting module is disposed on the wheel and is configured to be controlled to emit light with one of a plurality of color patterns. The light control module is disposed in the wheel and is electrically connected to the lighting module. The light control module includes a second wireless communication unit that is configured to receive the control signal from the first wireless communication module, and a light control unit that is configured to control the lighting module to emit light with one of the color patterns which corresponds to the corresponding one of the warning parameters included in the control signal. The power generating mechanism is disposed on the wheel and is configured to be driven by the wheel to generate electric power, where the electric power is to be provided to the lighting module and the light control module.

According to another aspect of the disclosure, a method for indicating a status of a driver of the vehicle body is to be implemented by the wheel system. With the target object being the driver, the method includes: the sensing module detecting the target object so as to generate the sensing data; in response to determining that one of the plurality of warning conditions is met based on the sensing data, the warning control module generating the control signal to include the corresponding one of the plurality of warning parameters; the light control module receiving the control signal from the warning control module through the second wireless communication unit, and identifying the corresponding one of the plurality of warning parameters that is included in the control signal; and the light control module controlling the lighting module to emit light with the one of the plurality of color patterns that corresponds to the corresponding one of the plurality of warning parameters included in the control signal.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a fragmentary schematic view illustrating a wheel system that is mounted on a vehicle according to an embodiment of the disclosure.

FIG. 2 is a perspective view illustrating components of a wheel assembly of the wheel system according to an embodiment of the disclosure.

FIG. 3 is a fragmentary schematic view illustrating a control device of the wheel system that is disposed in an inner space of the vehicle according to an embodiment of the disclosure.

FIG. 4 is a block diagram illustrating components of the wheel assembly and the control device according to an embodiment of the disclosure.

FIG. 5 is a flow chart illustrating steps of a method for indicating a status of a driver of a vehicle according to an embodiment of the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

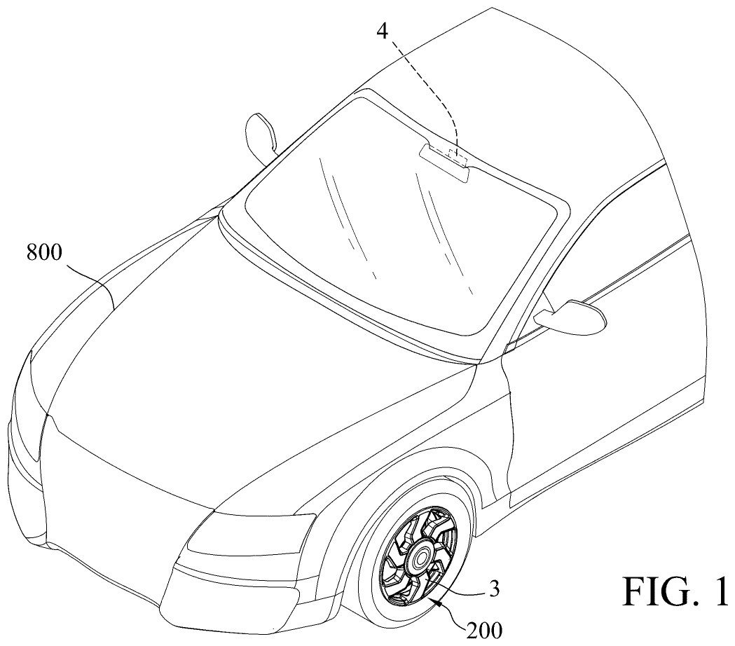

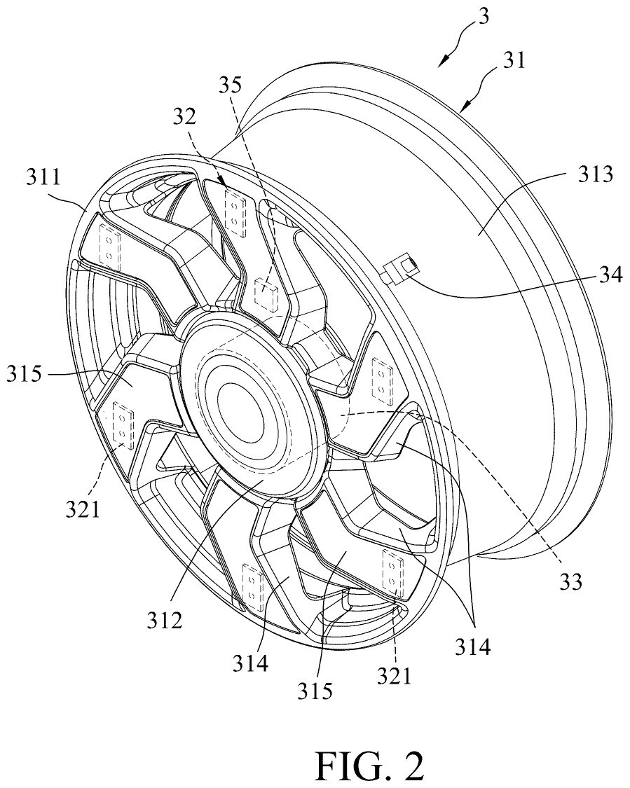

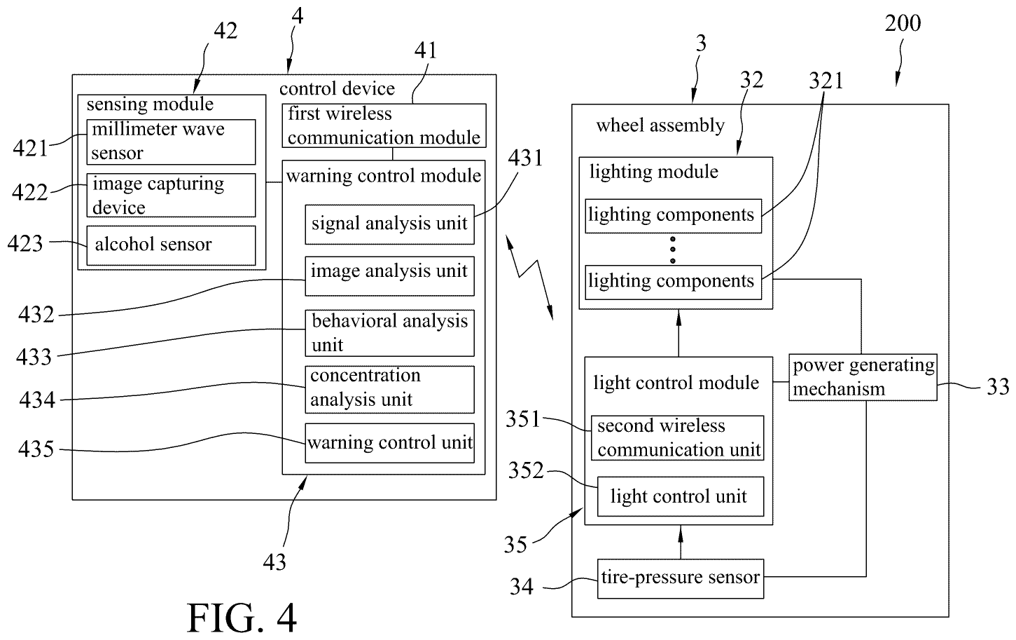

Referring to FIGS. 1 to 4, according to an embodiment of the disclosure, a wheel system 200 includes a wheel assembly 3 that is adapted to be mounted on a vehicle body 800 of a vehicle, and a control device 4 that is disposed in an inner space 801 of the vehicle body 800 and that is electrically connected to the wheel assembly 3. Specifically, a tire is mounted on the wheel assembly 3 to form a car wheel, and the car wheel is mounted on the vehicle body 800.

The control device 4 includes a first wireless communication module 41, a sensing module 42, and a warning control module 43 that is electrically connected to the first wireless communication module 41 and the sensing module 42. The first wireless communication module 41 is configured to wirelessly communicate with the wheel assembly 3 using wireless communication technology (e.g., Bluetooth®).

The sensing module 42 is configured to implement detection related to a target object in the inner space 801 of the vehicle body 800 so as to generate a sensing data set, and to send the sensing data set to the warning control module 43. The sensing module 42 includes a millimeter wave sensor 421, an image capturing device 422 and an alcohol sensor 423, and the sensing data set includes first sensing data generated by the millimeter wave sensor 421, second sensing data generated by the image capturing device 422, and third sensing data generated by the alcohol sensor 423. In this embodiment, the target object is a human body, and more particularly a driver of the vehicle.

The millimeter wave sensor 421 is configured to detect the target object (e.g., the driver) using millimeter wave radar technology so as to generate the first sensing data. Specifically, the millimeter wave sensor 421 is configured to emit millimeter wave signals at, for example, wavelengths ranging from 1 mm to 10 mm and frequencies ranging from 58 GHz to 62 GHZ, to the driver (i.e., the target object) who is sitting in the driver's seat of the vehicle, and to detect the millimeter wave signals reflected by the driver so as to generate the first sensing data.

The image capturing device 422 may be implemented by a camera that is configured to capture an image or a video of the driver (e.g., capturing the driver's seat or a specific area) so as to generate the second sensing data. The alcohol sensor 423 is configured to detect an alcohol content in air that is in the periphery of the driver (e.g., around the driver's seat) so as to generate the third sensing data. That is to say, the alcohol sensor 423 is used to measure the alcohol content in the air exhaled by the driver.

The warning control module 43 includes a signal analysis unit 431, an image analysis unit 432, a behavioral analysis unit 433, a concentration analysis unit 434 and a warning control unit 435, and stores a plurality of warning conditions, and a plurality of warning parameters corresponding respectively to the warning conditions.

The signal analysis unit 431 is configured to obtain a heart rate and a respiratory rate of the driver based on the first sensing data that is generated by the millimeter wave sensor 421. Methods for obtaining the heart rate and the respiratory rate based on millimeter wave signals is well known to one having ordinary skill in the art, and will not be described in further detail for the sake of brevity.

The image analysis unit 432 is configured to obtain a human body image portion (i.e., an image portion that includes the driver) from the second sensing data that is generated by the image capturing device 422 using image analysis technology.

The behavioral analysis unit 433 includes a behavioral determination model, and is configured to determine a behavior of the driver using the behavioral determination model based on the human body image portion. The behavioral determination model is related to, but not limited to, data collection and preprocessing, feature extraction, behavior analysis and categorization, behavior identification, time series analysis, risk determination, warning and interference.

Specifically, data collection and preprocessing includes collecting data with a sensor (not shown, e.g., an in-vehicle camera, an infrared camera, or a pressure sensor) that is connected to the behavioral analysis unit 433 or that is included in the sensing module 42, such as collecting facial features, head movement, or a line of sight of the driver using the sensor. Feature extraction includes obtaining facial features such as eye blink frequency, number of eye blinks, eye movement trajectories, mouth opening degree, head rotation angle, head rotation duration, head rotation frequency, and whether the driver's eyes are focused on the road or deviated for a long period of time. Behavior analysis and categorization includes analyzing the extracted facial features as mentioned above using a machine learning model, where the machine learning model may be a convolutional neural network (CNN) or a long short-term memory (LSTM) that is pre-trained. Behavior identification includes identifying whether the driver is in a state of fatigue, distracted, concentrated, etc. based on the analysis of the extracted facial features. Time series analysis includes analyzing changes in the extracted facial features over time, so as to determine whether there is an ongoing dangerous behavior by the driver. Risk determination includes setting different threshold values for the extracted facial features, respectively, so that when any of the extracted facial features exceeds the respective threshold value, the behavioral analysis unit 433 may determine that the driver is behaving dangerously. Warning includes obtaining a determination result on whether the behavior of the driver matches any of a plurality of preset dangerous behaviors, so as to determine whether a warning should be outputted, and outputting different levels of warnings (e.g., sound, vibration, visual alert) based on the determination result. In some embodiments, the wheel system 200 may be electrically connected to a central control system (not shown) of the vehicle, and interference includes the wheel system 200 and the central control system cooperatively interfering movement of the vehicle (e.g., by reducing speed or braking).

It should be noted that, the image analysis technology may include, but is not limited to, converting the image to grayscale, performing binary processing, and detecting the contours of various objects in the image, and then identifying the body image portion. The behavioral determination model may be trained by using algorithms to analyze and recognize images of various dangerous behaviors of a driver when driving a vehicle. The dangerous behaviors include, but are not limited to, viewing mobile phones, turning the head to the side for more than a predetermined length of time, and closing the eyes for more than a predetermined length of time, etc. The algorithms described in the disclosure may be computer vision algorithms using artificial intelligence (Al) and machine learning (ML), but the disclosure is not limited to such.

The concentration analysis unit 434 is configured to obtain an alcohol concentration value based on the third sensing data that is generated by the alcohol sensor 423.

The warning control unit 435 is configured to, in response to determining that one of the warning conditions is met based on the sensing data set, obtain a corresponding one of the warning parameters that corresponds to the one of the warning conditions, and output a control signal that includes the corresponding one of the warning parameters to the wheel assembly 3 through the first wireless communication module 41.

To describe in further detail, the warning control unit 435 is configured to, in response to determining that the heart rate obtained by the signal analysis unit 431 conforms with a first one of the warning conditions (e.g., the heart rate being greater than a predetermine rate, such as 110 beats per minute; hereinafter referred to as “first warning condition”), generate the control signal to include one of the warning parameters that corresponds to the first warning condition.

The warning control unit 435 is further configured to, in response to determining that the respiratory rate obtained by the signal analysis unit 431 conforms with a second one of the warning conditions (e.g., the respiration rate being greater than a predetermined rate, such as 25 breaths per minute; hereinafter referred to as “second warning condition”), generate the control signal to include one of the warning parameters that corresponds to the second warning condition.

It should be noted that the first warning condition for the heart rate and the second warning condition for the respiratory rate may be set by the driver, or may be set depending on the gender and/or age of the driver, but the disclosure is not limited to such.

The warning control unit 435 is further configured to, in response to determining that the behavior of the human body image portion (i.e., the behavior of the driver) determined by the behavioral analysis unit 433 conforms with a third one of the warning conditions (e.g., when the behavior of the driver conforms with one of the dangerous behaviors as mentioned above; hereinafter referred to as “third warning condition”), generate the control signal to include one of the warning parameters that corresponds to the third warning condition.

The warning control unit 435 is further configured to, in response to determining that the alcohol concentration value obtained by the concentration analysis unit 434 conforms with a fourth one of the warning conditions (e.g., the alcohol concentration value being greater than a predetermined value, such as 0.15 mg/L; hereinafter referred to as “fourth warning condition”), generate the control signal to include one of the warning parameters that corresponds to the fourth warning condition. It should be noted that the fourth warning condition may be set according to the traffic law of the country where the vehicle is being driven, and is not limited to the abovementioned example.

The warning control module 43 may be embodied using, but is not limited to, a system on a chip (SoC), a microcontroller, etc., and may include a central processing unit (CPU) and a memory that stores a software application. The software application includes instructions that, when executed by the CPU, cause the CPU to perform operations of the warning control module 43 as described above. In some embodiments, the CPU executing different portions of the software application may serve as the signal analysis unit 431, the image analysis unit 432, the behavioral analysis unit 433, the concentration analysis unit 434 and the warning control unit 435. In some embodiments, each unit of the warning control module 43 may be embodied using hardware elements, software elements, or a combination of both; example of hardware elements may include processors, microprocessors, circuits, integrated circuits, application specific integrated circuits (ASIC), programmable logic devices (PLD), digital signal processors (DSP), field programmable gate array (FPGA), logic gates, registers, semiconductor devices, chips, microchips, chip sets, and so forth.

The wheel assembly 3 includes a wheel 31 allowing the tire to be mounted thereon, and a lighting module 32, a power generating mechanism 33, a tire-pressure sensor 34 and a light control module 35 that are disposed on the wheel 31. The light control module 35 is electrically connected to the lighting module 32, the power generating mechanism 33 and the tire-pressure sensor 34.

The wheel 31 includes a wheel body 311 that is mounted on an axle (not shown) of the vehicle body 800, and a plurality of plates 315 that are disposed on the wheel body 311. The wheel body 311 includes a hub 312 that is mounted on the axle, a rim 313 that is radially spaced apart from and coaxially surrounding an outer peripheral surface of the hub 312, and a plurality of spokes 314 that are spaced apart from each other and that are connected between the outer peripheral surface of the hub 312 and an inner peripheral surface of the rim 313, where the outer peripheral surface of the hub 312 faces the inner peripheral surface of the rim 313. The plates 315 are disposed on the spokes 314, respectively, and are disposed on a side of the wheel body 311 that faces away from the vehicle.

The lighting module 32 includes a plurality of lighting components 321 that are disposed on the spokes 314 and that are covered by the plates 315. Each of the lighting components 321 is configured to be controlled by the light control module 35 to emit light with one of a plurality of color patterns through the plates 315. The lighting components 321 may each be a light-emitting diode (LED), but the disclosure is not limited to such. In this embodiment, the plates 315 are made of a translucent material. In some embodiments, the plates 315 may be replaced by any structure that allows the light emitted by the lighting components 321 to penetrate through, such as a plate formed with a light hole, but the disclosure is not limited to such.

The power generating mechanism 33 is coaxially disposed in the hub 312 and is driven by the wheel 31 to rotate so as to generate electric power, where the electric power is to be provided to components that are disposed on the wheel body 311, including the lighting module 32, the tire-pressure sensor 34 and the light control module 35. For details of the implementation of power generating mechanism 33, “the power generating device” disclosed in U.S. patent application Ser. No. 18/610,620 titled, “POWER GENERATING DEVICE,” “the electric power generating mechanism” disclosed in U.S. patent application Ser. No. 18/413,809 titled, “WHEEL RIM DEVICE,” and “the electric power generating mechanism” disclosed in U.S. patent application Ser. No. 18/413,816 titled, “WHEEL RIM DEVICE” may be referred to.

The tire-pressure sensor 34 is disposed on the rim 313 of the wheel body 311 and is configured to detect tire pressure of the tire mounted on the wheel 31 so as to obtain a tire pressure value.

The light control module 35 includes a second wireless communication unit 351 and a light control unit 352. The second wireless communication unit 351 is configured to wirelessly communicate with the first wireless communication module 41 of the control device 4 using wireless communication technology (e.g., Bluetooth®), and to wirelessly receive the control signal from the control device 4.

The light control unit 352 may be, but is not limited to, a microcontroller circuit. The light control unit 352 stores a plurality of color display settings, and a plurality of tire pressure conditions and the warning parameters. Each of the tire pressure conditions and the warning parameters corresponds to one of the color display settings. The light control unit 352 is configured to identify one of the tire pressure conditions that conforms with the tire pressure value detected by the tire-pressure sensor 34, to obtain a corresponding one of the color display settings that corresponds to the one of the tire pressure conditions, and to control the lighting components 321 to emit light with one of the color patterns based on the corresponding one of the color display settings.

In one example, Table 1 below shows standard front-tire and rear-tire pressure ranges for different vehicle models (generally around 35 psi). Specifically, the difference between the front-tire pressure range and the rear-tire pressure range of each vehicle model in Table 1 may be due to the difference in weights between the front portion and the rear portion of the vehicle model. In addition, the tire pressure may be affected by thermal expansion and contraction of air in the tires due to seasonal factors (e.g., during winter or summer). Therefore, the values set for the tire pressure conditions should account for the abovementioned factors. Generally speaking, to accommodate various vehicle models and scenarios, the tire pressure conditions in this disclosure is set so that, for example, when the tire pressure is higher than 45 psi or lower than 25 psi (i.e., when the tire pressure is too high or too low), the lighting components 321 are controlled to emit red light as a warning. That is to say, the color display setting that corresponds to the tire pressure condition of higher than 45 psi is red light, and the color display setting that corresponds to the tire pressure condition of lower than 25 psi is also red light. However, the disclosure is not limited to such.

TABLE 1

| | Front-Tire Pressure | Rear-Tire Pressure |
|---|---|---|
| Vehicle Model | Ranges (psi) | Ranges (psi) |
| Honda City | 32-35 | 30-33 |
| Toyota Camry | 33-36 | 32-35 |
| Ford Taurus | 35-38 | 33-36 |
| Nissan Altima | 33-36 | 32-35 |
| Volkswagen Jetta | 32-35 | 30-33 |
| Hyundai Elantra | 32-35 | 30-33 |
| BMW 3 Series | 35-38 | 33-36 |
| Audi A4 | 35-38 | 33-36 |

The light control unit 352 is further configured to obtain one of the warning parameters that is included in the control signal received by the second wireless communication unit 351, to obtain a corresponding one of the color display settings that corresponds to the one of the warning parameters thus obtained, and to control the lighting components 321 to emit light with one of the color patterns based on the corresponding one of the color display settings.

In this embodiment, the light control module 35 is configured to first control the lighting components 321 to emit light with one of the color patterns based on the tire pressure value, and in response to receiving the control signal that includes one of the warning parameters, control the lighting components 321 to emit light with one of the color patterns based on the control signal instead. In some embodiments, the light control module 35 is configured to control the lighting components 321 to emit light based on the tire pressure value and the control signal in an alternative manner. However, the disclosure is not limited to the abovementioned examples.

The wheel system 200 of the disclosure is to be mounted on the vehicle body 800, and while the vehicle is moving, the power generating mechanism 33 of the wheel assembly 3 will be driven to rotate so as to generate electric power for the wheel assembly 3. The tire-pressure sensor 34 continuously obtains the tire pressure value, and the light control module 35 identifies one of the tire pressure conditions that conforms with the tire pressure value obtained by the tire-pressure sensor 34, and controls the lighting components 321 to emit light with one of the color patterns based on one of the color display settings that corresponds to the one of the tire pressure conditions. As such, the wheel 31 may emit light to warn people or vehicles in the surrounding area.

Moreover, the millimeter wave sensor 421, the image capturing device 422 and the alcohol sensor 423 of the control device 4 implement detection related to the driver (or the driver's seat) to generate the sensing data set. In response to determining that one of the warning conditions is met based on the sensing data set, the warning control module 43 generates the control signal to include one of the warning parameters that corresponds to the one of the warning conditions, and outputs the control signal to the second wireless communication unit 351 through the first wireless communication module 41.

Then, the light control module 35 of the wheel assembly 3 controls the lighting components 321 to emit light with one of the color patterns based on the control signal, so that the wheel 31 may emit light to warn people or vehicles in the surrounding area.

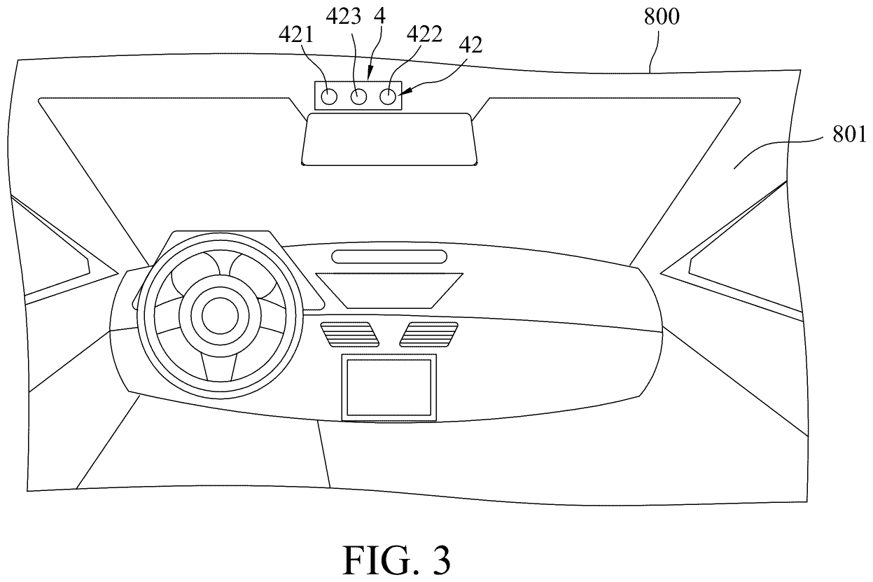

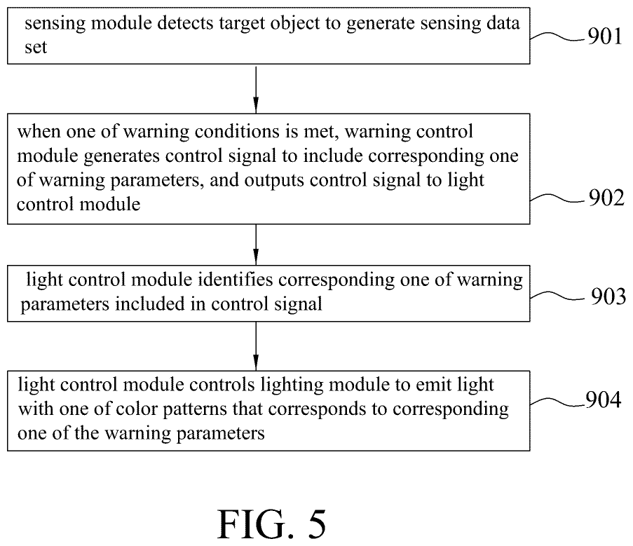

Referring to FIGS. 3 to 5, a method for indicating a status of the driver of the vehicle according to an embodiment of the disclosure is provided. The method is to be implemented by the wheel system 200 that is mounted on the vehicle body 800, and is used for detecting the status of the driver and outputting warning when the status of the driver is abnormal. The method includes steps 901 to 904.

In step 901, the sensing module 42 of the control device 4 implements the detection related to the target object (e.g., the driver or the driver's seat) to generate the sensing data set. The sensing data set is related to at least one of a heart rate, a respiration rate, an alcohol concentration, an eye blink frequency, a number of eye blinks, an eye movement trajectory, a mouth opening degree, a head rotation angle, a head rotation duration, a head rotation frequency, whether the driver's eyes are focused on a road, or whether the driver's eyes are deviated from the road for a long period of time.

In step 902, in response to determining that one of the warning conditions is met based on the sensing data set, the warning control module 43 generates the control signal to include the corresponding one of the warning parameters, and outputs the control signal to the light control module 35.

In step 903, the light control module 35 receives the control signal from the warning control module 43, and identifies the corresponding one of the warning parameters that is included in the control signal.

In step 904, the light control module 35 controls the lighting module 32 to emit light with one of the color patterns (e.g., red light) that corresponds to the corresponding one of the warning parameters included in the control signal, so that people or vehicles in the surrounding area may be warned by the emitted light.

In summary, according to the disclosure, the wheel system 200 is capable of, when the vehicle is moving, implementing the detection related to the target object (e.g., the driver) in the inner space 801 so as to generate the sensing data set, and controlling the wheel assembly 3 to emit light with one of the color patterns when determining that one of the warning conditions is met based on the sensing data set. As such, people and vehicles in the surrounding area may be warned by the emitted light to stay away from the vehicle, and thus reducing chances of traffic accidents happening. Specifically, the people mentioned above may be pedestrians or drivers of other vehicles, motorcycles, or bicycles.

In one embodiment, each of the first wireless communication module 41 and the second wireless communication unit 351 uses an ultra-wide band (UWB) wireless communication technology that operates in a frequency range from 31 MHz to 88 MHz, and a ratio of a bandwidth of the circuit to a center frequency of the circuit is greater than 20%. Since this frequency range is a spectrum typically dedicated to the army, and the center frequency is related to the sensitivity of the sensor and the maximum penetration depth of the emitted UWB signal, this frequency range may be applied to vehicles with thicker armor, such as military trucks, armored vehicles and wheeled vehicles.

In another embodiment, each of the first wireless communication module 41 and the second wireless communication unit 351 uses an ultra-wide band (UWB) wireless communication technology that operates in a frequency range from 3.1 GHz to 10.6 GHZ, and a ratio of a bandwidth of the circuit to a center frequency of the circuit is greater than 10%. Since this frequency range is a spectrum typically used by civilian, and the center frequency is related to the resolution of the sensor and affects the size of the wireless receiver, this frequency range may be applied to vehicles that are generally not affected by the size of the antenna and the receiving module, such as civilian cars, trucks and sports utility vehicles.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features.

In other words, in any described embodiment, when implementation of one or more features or specific details does not affect implementation of another one or more features or specific details, said one or more features may be singled out and practiced alone without said another one or more features or specific details.

It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A wheel system for a vehicle body, comprising:
a control device including
a first wireless communication module,
a sensing module that is configured to implement detection related to a target object in the vehicle body so as to generate sensing data, and
a warning control module that stores a plurality of warning conditions, and a plurality of warning parameters corresponding respectively to the plurality of warning conditions, and that is configured to, in response to determining that one of the plurality of warning conditions is met based on the sensing data, obtain a corresponding one of the plurality of warning parameters that corresponds to the one of the plurality of warning conditions, and output a control signal that includes the corresponding one of the plurality of warning parameters through said first wireless communication module; and
a wheel assembly adapted to be mounted on the vehicle body and including
a wheel,
a lighting module that is disposed on said wheel and that is configured to be controlled to emit light with one of a plurality of color patterns,
a light control module that is disposed in said wheel, that is electrically connected to said lighting module, and that includes a second wireless communication unit configured to receive the control signal from said first wireless communication module, and a light control unit configured to control said lighting module to emit light with one of the plurality of color patterns which corresponds to the corresponding one of the plurality of warning parameters included in the control signal, and
a power generating mechanism that is disposed on said wheel and that is configured to be driven by said wheel to generate electric power, where the electric power is to be provided to said lighting module and said light control module.

2. The wheel system as claimed in claim 1, the target object being a human body,
wherein said sensing module includes a millimeter wave sensor that is configured to detect the target object using millimeter wave radar technology so as to generate the sensing data, and
wherein said warning control module includes a signal analysis unit that is configured to obtain a heart rate based on the sensing data, and a warning control unit that is configured to, in response to determining that the heart rate conforms with the one of the plurality of warning conditions, generate the control signal which includes the corresponding one of the plurality of warning parameters.

3. The wheel system as claimed in claim 1, the target object being a human body,
wherein said sensing module includes a millimeter wave sensor that is configured to detect the target object using millimeter wave radar technology so as to generate the sensing data, and
wherein said warning control module includes a signal analysis unit that is configured to obtain a respiratory rate based on the sensing data, and a warning control unit that is configured to, in response to determining that the respiratory rate conforms with the one of the plurality of warning conditions, generate the control signal which includes the corresponding one of the plurality of warning parameters.

4. The wheel system as claimed in claim 1, the target object being a human body,
wherein said sensing module includes an image capturing device that is configured to capture an image of the target object so as to generate the sensing data, and
wherein said warning control module includes an image analysis unit that is configured to obtain a human body image portion from the sensing data using image analysis technology, a behavioral analysis unit that includes a behavioral determination model and that is configured to determine a behavior of the human body image portion based on the behavioral determination model, and a warning control unit that is configured to, in response to determining that the behavior of the human body image portion conforms with the one of the plurality of warning conditions, generate the control signal which includes the corresponding one of the plurality of warning parameters.

5. The wheel system as claimed in claim 1, wherein:
said sensing module includes an alcohol sensor that is configured to detect an alcohol content in air inside the vehicle body so as to generate the sensing data, and
said warning control module includes a concentration analysis unit that is configured to obtain an alcohol concentration value based on the sensing data, and a warning control unit that is configured to, in response to determining that the alcohol concentration value conforms with the one of the plurality of warning conditions, generate the control signal which includes the corresponding one of the plurality of warning parameters.

6. The wheel system as claimed in claim 1, wherein said wheel allows a tire to be mounted thereon, and said wheel assembly includes a tire-pressure sensor that is installed on said wheel and that is configured to detect tire pressure of the tire so as to obtain a tire pressure value,
wherein said light control module stores a plurality of tire pressure conditions and a plurality of color display settings, where each of the plurality of tire pressure conditions corresponds to one of the plurality of color display settings, and is configured to identify one of the plurality of tire pressure conditions that conforms with the tire pressure value, obtain a corresponding one of the plurality of color display settings that corresponds to the one of the plurality of tire pressure conditions, and control said lighting module to emit light with one of the plurality of color patterns based on the corresponding one of the plurality of color display settings.

7. The wheel system as claimed in claim 1, wherein said wheel includes a wheel body, said wheel body including a hub, a rim that is radially spaced apart from and coaxially surrounding an outer peripheral surface of said hub, and a plurality of spokes that are spaced apart from each other and that are connected between the outer peripheral surface of said hub and an inner peripheral surface of said rim, the outer peripheral surface of said hub facing the inner peripheral surface of said rim,
wherein said power generating mechanism is coaxially disposed in said hub, and
wherein said lighting module includes a lighting component that is disposed on one of said plurality of spokes and that is configured to be controlled to emit light with one of a plurality of color patterns.

8. The wheel system as claimed in claim 7, wherein said lighting module includes a plurality of lighting components that are disposed on said plurality of spokes.

9. The wheel system as claimed in claim 8, wherein said wheel further includes a plurality of plates that are disposed on said plurality of spokes and that cover said plurality of lighting components, where said plurality of plates allow the light emitted by said plurality of lighting components to penetrate through.

10. A method for indicating a status of a driver of the vehicle body to be implemented by the wheel system as claimed in claim 1, the target object being the driver, the method comprising:
the sensing module detecting the target object so as to generate the sensing data;
in response to determining that one of the plurality of warning conditions is met based on the sensing data, the warning control module generating the control signal to include the corresponding one of the plurality of warning parameters;
the light control module receiving the control signal from the warning control module through the second wireless communication unit, and identifying the corresponding one of the plurality of warning parameters that is included in the control signal; and
the light control module controlling the lighting module to emit light with the one of the plurality of color patterns that corresponds to the corresponding one of the plurality of warning parameters included in the control signal.

11. The method as claimed in claim 10, the sensing module including a millimeter wave sensor that is configured to detect the target object using millimeter wave radar technology so as to generate the sensing data, the method comprising:
the warning control module obtaining a heart rate based on the sensing data, and in response to determining that the heart rate conforms with the one of the plurality of warning conditions, generating the control signal to include the corresponding one of the plurality of warning parameters;
the light control module receiving the control signal from the warning control module through the second wireless communication unit, and identifying the corresponding one of the plurality of warning parameters that is included in the control signal; and
the light control module controlling the lighting module to emit light with the one of the plurality of color patterns that corresponds to the corresponding one of the plurality of warning parameters included in the control signal.

12. The method as claimed in claim 10, the sensing module including a millimeter wave sensor that is configured to detect the target object using millimeter wave radar technology so as to generate the sensing data, the method comprising:
the warning control module obtaining a respiratory rate based on the sensing data, and in response to determining that the respiratory rate conforms with the one of the plurality of warning conditions, generating the control signal to include the corresponding one of the plurality of warning parameters;
the light control module receiving the control signal from the warning control module through the second wireless communication unit, and identifying the corresponding one of the plurality of warning parameters that is included in the control signal; and
the light control module controlling the lighting module to emit light with the one of the plurality of color patterns that corresponds to the corresponding one of the plurality of warning parameters included in the control signal.

13. The method as claimed in claim 10, the sensing module including an image capturing device that is configured to capture an image of the target object so as to generate the sensing data, the method comprising:
the warning control module obtaining a human body image portion from the sensing data using the image analysis technology, determining a behavior of the human body image portion based on a behavioral determination model, and in response to determining that the behavior of the human body image portion conforms with the one of the plurality of warning conditions, generating the control signal to include the corresponding one of the plurality of warning parameters;
the light control module receiving the control signal from the warning control module through the second wireless communication unit, and identifying the corresponding one of the plurality of warning parameters that is included in the control signal; and
the light control module controlling the lighting module to emit light with the one of the plurality of color patterns that corresponds to the corresponding one of the plurality of warning parameters included in the control signal.

14. The method as claimed in claim 10, the sensing module including an alcohol sensor that is configured to detect an alcohol content in air inside the vehicle body so as to generate the sensing data, the method comprising:
the warning control module obtaining an alcohol concentration value based on the sensing data, and in response to determining that the alcohol concentration value conforms with the one of the plurality of warning conditions, generating the control signal to include the corresponding one of the plurality of warning parameters;
the light control module receiving the control signal from the warning control module through the second wireless communication unit, and identifying the corresponding one of the plurality of warning parameters that is included in the control signal; and
the light control module controlling the lighting module to emit light with the one of the plurality of color patterns that corresponds to the corresponding one of the plurality of warning parameters included in the control signal.
